(19) United States
(12) Patent Application Publication
(10) Pub. No.: US 2025/0004214 A1
(43) Pub. Date: Jan. 2, 2025

(54) OPTICAL TRANSMISSION SYSTEM

(71) Applicant: Nippon Telegraph and Telephone Corporation, Tokyo (JP)

(72) Inventors: Takahito KIRIHARA, Musashino-shi, Tokyo (JP); Satoshi Narikawa, Musashino-shi, Tokyo (JP); Katsuhisa TAGUCHI, Musashino-shi, Tokyo (JP); Ayako IWAKI, Musashino-shi, Tokyo (JP); Kazuhide Nakajima, Musashino-shi, Tokyo (JP); Takashi Matsui, Musashino-shi, Tokyo (JP); Chisato Fukai, Musashino-shi, Tokyo (JP); Yuto SAGAE, Musashino-shi, Tokyo (JP); Tomohiro TANIGUCHI, Musashino-shi, Tokyo (JP); Hiroyuki IIDA, Musashino-shi, Tokyo (JP)

(21) Appl. No.: 18/707,816

(22) PCT Filed: Nov. 10, 2022
(86) PCT No.: PCT/JP2022/042007

§ 371 Date: May 6, 2024

(30) Foreign Application Priority Data
Nov. 11, 2021 (WO) PCT/JP2021/041492

(51) Int. Cl. G02B 6/42; A61L 2/10; A61L 2/24; A61L 2/26

(52) U.S. Cl. CPC G02B 6/4206; A61L 2/10; A61L 2/24; A61L 2/26; A61L 2202/11; A61L 2202/14

(57) ABSTRACT

An object of the present invention is to provide an optical transmission system capable of reducing system costs and reducing waste of output power of a light source. The optical transmission system according to the present invention includes an ultraviolet light source part 11a having a light emitting diode (LED) that outputs ultraviolet light, an optical transmission line 26 that propagates the ultraviolet light through a plurality of cores of a multi-core optical fiber or a plurality of cores of a bundle optical fiber obtained by bundling a plurality of single-core optical fibers, and an optical system 11c that narrows down a spot shape of the ultraviolet light output from the LED to a size including all of the plurality of cores in an optical axis direction, and causes the ultraviolet light to enter the plurality of cores.

### TECHNICAL FIELD

The present disclosure relates to an optical transmission system having a point-to-multipoint (P-MP) configuration for propagating light from one light source to a plurality of irradiation target regions.

### BACKGROUND ART

For the purpose of preventing infectious diseases, there is an increasing demand for systems for sterilization and inactivation of viruses using ultraviolet light. Systems have three major categories of products. In the present specification, when the term “sterilization or the like” is used, it means sterilization and inactivation of viruses.

### (I) Mobile Sterilization Robot

The product of NPL 1 is an autonomous mobile robot for emitting ultraviolet light. The robot emits ultraviolet light while moving in a room in a building such as a hospital room, thereby automatically achieving sterilization or the like over a wide range without human intervention.

### (II) Stationary Air Purifier

The product of NPL 2 is a device that is installed on a ceiling or at a predetermined place in a room, and performs sterilization or the like while circulating the air in the room. Since the device does not directly emit ultraviolet light and does not affect the human body, highly safe sterilization or the like can be performed.

### (III) Portable Sterilization Device

The product of NPL 3 is a portable device equipped with an ultraviolet light source. A user can transport the device to a desired area and irradiate the area with ultraviolet light. Thus, the device can be used in various places.

### CITATION LIST

### Non Patent Literature

[NPL 1] Kantum Ushikata Co., Ltd. website (https://www.kantum.co.jp/product/sakkin_robot/sakkinn_robot/U VD_robot)
[NPL 2] IWASAKI ELECTRIC CO., Ltd. website (https://www.iwasaki.co.jp/optics/sterilization/air/air03.html
[NPL 3] Funakoshi Co., Ltd. website (https://www.funakoshi.co.jp/contents/68182)

### SUMMARY OF INVENTION

### Technical Problem

However, the devices described in NPL 1 to NPL 3 have the following problems.

### (1) Economic Efficiency

The product of NPL 1 emits high-output ultraviolet light, and thus the device becomes large-scaled and expensive. Therefore, the product of NPL 1 has a problem that it is difficult to achieve an economical system.

### (2) Versatility

In the product of NPL 1, since the ultraviolet light irradiation site is limited to a place where the robot can move/enter, it is difficult to emit the ultraviolet light to small places or recessed places.

Since the product of NPL 2 sterilizes the circulated indoor air or the like, it is not possible to directly emit ultraviolet light to a place to be sterilized.

The product of NPL 3, for example, cannot emit ultraviolet light to narrow pipes or areas where people cannot enter. Thus, the products of NPL 1 to NPL 3 have a problem in versatility of being able to emit ultraviolet light to any place.

### (3) Operability

The product of NPL 3 is portable and can emit ultraviolet light at various places. However, in order to obtain a sufficient effect such as sterilization at a target location, a user is required to have skills or knowledge, and there is a problem in operability.

To solve these problems, an ultraviolet light irradiation system 300 using an optical fiber as illustrated in FIG. 1 can be considered. The ultraviolet light irradiation system transmits ultraviolet light from a light source part 11 using a thin and easily bendable optical fiber, and irradiates an irradiation target region AR to be sterilized or the like with ultraviolet light output from the tip of an optical fiber 14 with a pinpoint. Since any place can be irradiated with ultraviolet light simply by moving an irradiation part 13 at the tip of the optical fiber 14, the versatility of the above-mentioned problem (2) can be solved. In addition, since there is no need to move or set the ultraviolet light source, and the user is not required to have skills or knowledge, the operability of the above-mentioned problem (3) can be solved. Further, by providing an optical distribution part 12 such as an optical splitter in an optical transmission line 16 to form a point-to-multipoint (P-MP) system configuration such as fiber to the home (FTTH), a plurality of locations can be sterilized or the like by sharing a single light source. Therefore, the economic efficiency of the above-mentioned problem (1) can also be solved.

On the other hand, there are problems as illustrated in FIGS. 2 and 3 in achieving a P-MP configuration as an ultraviolet light irradiation system. The optical transmission line 16 is typically a single-core optical fiber. The single-core optical fiber has a small core area in cross section.

FIG. 2 is a diagram for describing a case where the light source part 11 is a laser. The output beam of the laser can be easily focused by an optical system 11c, and ultraviolet light can efficiently enter a core of a narrow area of an optical fiber of the optical transmission line 16. However, lasers are expensive, and there is a problem that it is difficult to reduce system costs.

FIG. 3 is a diagram for describing a case where the light source part 11 is a light emitting diode (LED). The LED is inexpensive compared with the laser, and the system cost can be reduced. However, since the LED has a large light-emitting surface, output light is not sufficiently focused even if the optical system 11c is used, and it is difficult to cause ultraviolet light to efficiently enter a core of a narrow area of an optical fiber of the optical transmission line 16. On the other hand, when the core area of the optical fiber is increased, the allowable bending radius is also increased, and the degree of freedom of routing at the time of laying the optical fiber is limited. In other words, when the LED is used for the light source part 11, the core area of the optical fiber cannot be enlarged in consideration of the degree of freedom of routing at the time of laying the optical fiber, and there is a problem that it is difficult to effectively utilize the output power of the light source (there is a lot of waste).

The above-mentioned problems are not limited to ultraviolet light, and are the same for infrared light and visible light. Accordingly, in order to solve the problems, it is an object of the present invention to provide an optical transmission system capable of reducing system costs and reducing waste of output power of a light source.

### Solution to Problem

In order to achieve the above object, the optical transmission system according to the present invention uses a light source such as an LED as a light source part, and propagates light from the light source part by a spatial multiplex transmission method such as a bundled single-core optical fiber or multi-core optical fiber.

Specifically, an optical transmission system according to the present invention includes:
a light source part that outputs light;
an optical transmission line that propagates the light through a plurality of cores of a multi-core optical fiber or a plurality of cores of a bundle optical fiber obtained by bundling a plurality of single-core optical fibers; and an optical system that causes the light output from the light source part to enter the plurality of cores, and
the optical system arbitrarily adjusts a coupling state in which the light enters the core.

The optical system narrows down a spot shape of the light to a size including all of the plurality of cores in an optical axis direction as the adjustment of the coupling state.

Here, the light source part may be an ultraviolet light source part having a light emitting diode (LED) that outputs ultraviolet light as the light. The optical system may be configured to narrow down a spot shape of the ultraviolet light output from the LED to a size including all the plurality of cores in an optical axis direction, and to cause the ultraviolet light to enter the plurality of cores.

Since this optical transmission system employs a light source such as an LED as the light source part, the system cost can be reduced compared with an optical transmission system employing a laser as the light source part.

In addition, this optical transmission system employs a multi-core optical fiber or a bundle optical fiber (a bundle of a plurality of single-core optical fibers) as an optical transmission line. Since the light source part is a light source such as an LED, the spot shape of the ultraviolet light cannot be narrowed down to the diameter of a single core by an optical system, but the waste of the output power of the light source part can be reduced by causing the light which has not been narrowed down to enter a plurality of cores disposed in a light collecting region.

Therefore, the present invention can provide an optical transmission system capable of reducing system costs and reducing waste of output power of the light source part.

The optical transmission system according to the present invention further includes irradiation parts that irradiate a plurality of irradiation target regions with the light propagated through the plurality of cores, respectively. When the light source part side of the optical transmission line is taken as one end, an optical distribution part (fan-out device) is disposed at the other end of the optical transmission line, and a P-MP configuration for delivering and emitting light to a plurality of irradiation target regions can be employed.

The optical transmission system according to the present invention further includes a monitoring part that monitors propagation of the light with respect to at least one of the cores.

When the propagating light is light other than visible light, it is difficult to visually check whether or not the light is being propagated. Therefore, by installing a monitoring part capable of detecting light in any of the cores, it is possible to easily ascertain whether the optical transmission system is in operation (in a state in which light is being propagated) or idle (in a state in which light is not being propagated).

It is preferable that the core monitored by the monitoring part be the core disposed on an outer periphery in a cross section of the optical transmission line.

Even if the coupling state is adjusted by the optical system, it is difficult to obtain uniform power in all the cores of the optical transmission line, and power deviation usually occurs. For example, in the cross section of the optical transmission line, although light having high power propagates through the cores near the center, light having low power propagates through the cores of the outer peripheral portion. The light propagating through the core of the outer peripheral portion has low power and may not be usable at the light irradiation destination (irradiation target region) (for example, when the light is ultraviolet light, the power is too small to sufficiently inactivate the irradiation target region).

Therefore, if monitoring is performed using the light propagating through the core of the outer peripheral portion, which cannot be used in the irradiation target region, it is possible to effectively use the light that has been propagated wastefully.

The optical transmission system according to the present invention further includes a control part that performs feedback control on at least one of power of the light output from the light source part and the coupling state adjusted by the optical system so that power of the light monitored by the monitoring part becomes a desired value.

By performing feedback control by the control part, it is possible to supply light of a power desired for the irradiation target region.

Here, the control part can perform the feedback control using at least one of the cores. Further, it is preferable that the core used for the feedback control be the core disposed on the outer periphery in a cross section of the optical transmission line.

The feedback control may be performed by wire or wirelessly, or may be performed by optical communication using the core of the optical transmission line. In this case, if the core of the outer peripheral portion of the optical transmission line that cannot be used in the irradiation target region due to the low light power is used for optical communication as described above, the resources can be effectively used.

Note that the above inventions can be combined to any extent possible.

### Advantageous Effects of Invention

The present invention can provide an optical transmission system capable of reducing system costs and reducing waste of output power of a light source.

### BRIEF DESCRIPTION OF DRAWINGS

FIG. 1 is a diagram illustrating problems of the present invention.

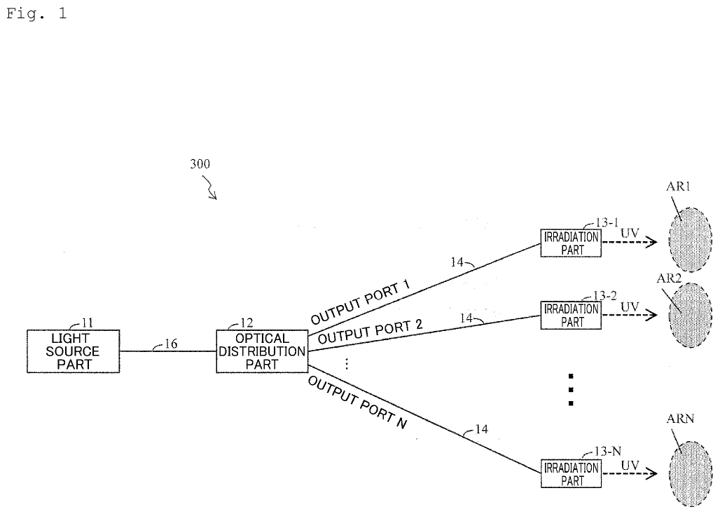

FIG. 2 is a diagram illustrating the problems of the present invention.

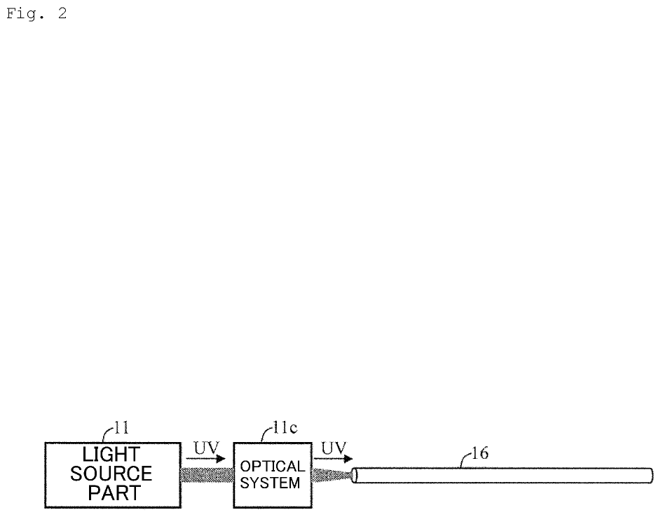

FIG. 3 is a diagram illustrating the problems of the present invention.

FIG. 4 is a diagram illustrating an optical transmission system according to the present invention.

FIG. 5 is a diagram illustrating an optical transmission line of an optical transmission system according to the present invention.

FIG. 6 is a drawing illustrating a cross-sectional structure of an optical fiber.

FIG. 7 is a diagram illustrating a state in which light enters an optical fiber of an optical transmission system according to the present invention.

FIG. 8 is a diagram illustrating an optical transmission system according to the present invention.

FIG. 9 is a diagram illustrating the problems of the present invention.

FIG. 10 is a diagram illustrating an optical transmission system according to the present invention.

FIG. 11 is a diagram illustrating an optical transmission system according to the present invention.

FIG. 12 is a diagram illustrating an optical transmission system according to the present invention.

### DESCRIPTION OF EMBODIMENTS

Embodiments of the present invention will be described with reference to the accompanying drawings. The embodiments described below are examples of the present invention, and the present invention is not limited to the following embodiments. Constituent elements with the same reference numerals in the present specification and the drawings represent the same constituent elements. Further, in the present embodiment, a case where a light source part outputs ultraviolet light will be described, but the same is true for a case where the light source part outputs infrared light or visible light.

### Embodiment 1

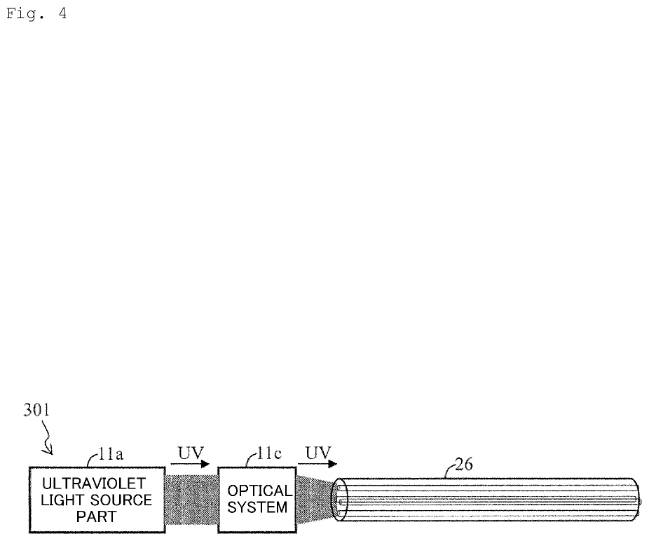

FIG. 4 is a diagram illustrating an optical transmission system 301 of the present embodiment. The optical transmission system 301 includes
an ultraviolet light source part 11a having a light emitting diode (LED) that outputs ultraviolet light,
an optical transmission line 26 that propagates the ultraviolet light through a plurality of cores of a multi-core optical fiber or a plurality of cores of a bundle optical fiber obtained by bundling a plurality of single-core optical fibers, and
an optical system 11c that narrows down a beam diameter of the ultraviolet light output from the LED to a diameter of a circle including all of the plurality of cores in an optical axis direction, and causes the ultraviolet light to enter the plurality of cores.

The ultraviolet light source part 11a includes an LED, and outputs light in an ultraviolet region (ultraviolet light) that is effective for sterilization or the like with the LED. The optical system 11c collects the ultraviolet light output from the ultraviolet light source part 11a to reduce the beam diameter. For example, the optical system 11c is a lens designed for wavelengths in the ultraviolet region.

The optical system 11c is not limited to narrowing down the beam diameter of a circular shape on a plane perpendicular to the optical axis up to the diameter of a circle including all of the plurality of cores in the optical axis direction. When the shape of light emitted from the ultraviolet light source part 11a on a plane perpendicular to the optical axis is a spot shape other than a circle (for example, an elliptical shape, a polygonal shape), the optical system 11c also narrows down the light to a size including all of the plurality of cores in the optical axis direction.

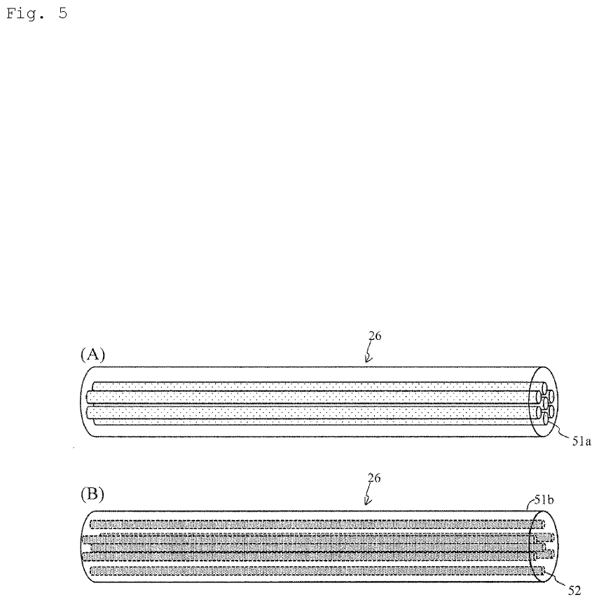

FIG. 5 is a diagram illustrating a structure of the optical transmission line 26. The optical transmission line 26 is a bundle optical fiber obtained by bundling single-core optical fibers 51a as illustrated in FIG. 5(A) or a multi-core optical fiber 51b having a plurality of cores 52 as illustrated in FIG. 5(B).

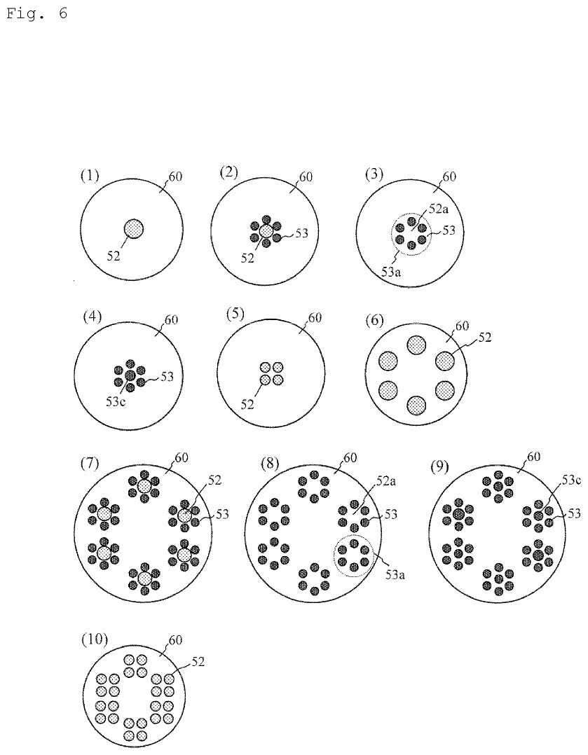

FIG. 6 is a diagram illustrating a cross section of an optical fiber. As the single-core optical fiber 51a described above, optical fibers having a cross-sectional structure as illustrated in (1) to (5) in FIG. 6 can be used.

### (1) Solid Core Optical Fiber

This optical fiber has one solid core 52 having a refractive index higher than that of a clad 60 in the clad 60. “Solid” means “not hollow.” The solid core can also be realized by forming an annular low refractive index region in the clad.

### (2) Hole-Assisted Optical Fiber

This optical fiber has a solid core 52 and a plurality of holes 53 disposed on the outer periphery of the solid core 52 in a clad 60. The medium of the hole 53 is air, and the refractive index of the air is sufficiently smaller than that of quartz glass. Therefore, the hole-assisted optical fiber has a function of returning light leaking from the core 52 by bending or the like to the core 52 again, and has a characteristic of small bending loss.

### (3) Hole-Structured Optical Fiber

This optical fiber has a hole group 53a of a plurality of holes 53 in a clad 60, and has a refractive index effectively lower than that of a host material (glass or the like). This structure is called a photonic crystal fiber. This structure can have a structure in which a high refractive index core having a changed refractive index is not present, and light can be confined by making a region 52a surrounded by the holes 53 an effective core region. As compared with an optical fiber having a solid core, the photonic crystal fiber can reduce the influence of absorption and scattering loss of the core by an additive, and can realize optical characteristics which cannot be realized by the solid optical fiber such as reduction of bending loss and control of nonlinear effect.

### (4) Hollow Core Optical Fiber

The core region of this optical fiber is formed of air. Light can be confined in the core region by taking a photonic band gap structure by a plurality of holes or an anti-resonant structure by a glass thin wire in the cladding region. The optical fiber has a small nonlinear effect and can supply a high output or high energy laser.

### (5) Coupled Core Type Optical Fiber

In this optical fiber, a plurality of solid cores 52 having a high refractive index are disposed in close proximity to each other in a clad 60. The optical fiber guides light by light wave coupling between the solid cores 52.

Since the light can be dispersed and sent by the number of cores, there is an advantage that the power can be increased by that amount and efficient sterilization or the like can be performed, or the deterioration of the fiber due to ultraviolet light can be relaxed and the service life can be prolonged.

As the multi-core optical fiber 51b described above, optical fibers having a cross-sectional structure as illustrated in (6) to (10) in FIG. 6 can be used.

### (6) Solid Core Type Multi-Core Optical Fiber

In this optical fiber, a plurality of solid cores 52 having a high refractive index are disposed apart from each other in a clad 60.

This optical fiber guides light in a state where the influence of the light wave coupling can be ignored by sufficiently reducing the light wave coupling between the solid cores 52. There is an advantage that each core can be treated as an independent waveguide.

### (7) Hole-Assisted Type Multi-Core Optical Fiber

This optical fiber has a structure in which a plurality of the hole structures and core regions of the above (2) are disposed in a clad 60.

### (8) Hole-Structured Type Multi-Core Optical Fiber

This optical fiber has a structure in which a plurality of the hole structures of the above (3) are disposed in a clad 60.

### (9) Hollow Core Type Multi-Core Optical Fiber

This optical fiber has a structure in which a plurality of the hole structures of the above (4) are disposed in a clad 60.

### (10) Coupled Core Type Multi-Core Optical Fiber

This optical fiber has a structure in which a plurality of the coupled core structures of the above (5) are disposed in a clad 60.

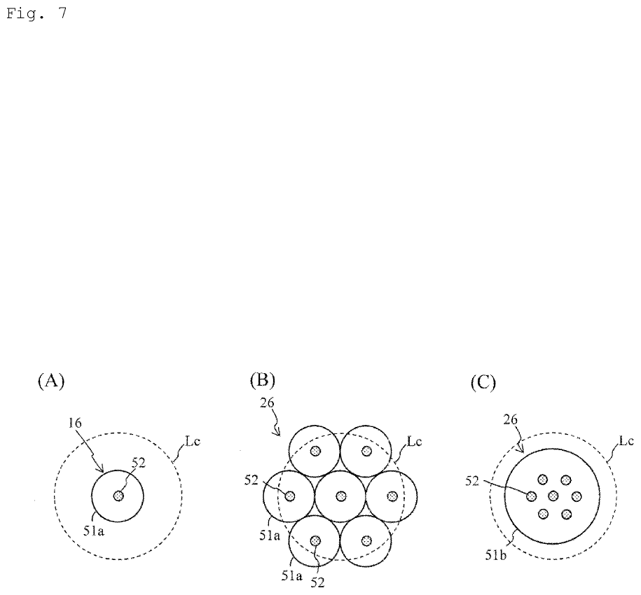

FIG. 7 is a diagram illustrating a state in which ultraviolet light enters one end of the optical transmission line (16, 26) from the optical system 11c. In FIG. 7, one end of the optical transmission line (16, 26) is viewed from the optical system 11c side. As described with reference to FIG. 6, there are various core structures, but here, the solid core 52 will be described as a representative. In FIG. 7, a reference numeral Lc denotes a light collecting region in which the optical system 11c can collect ultraviolet light at one end of the optical transmission line (16, 26).

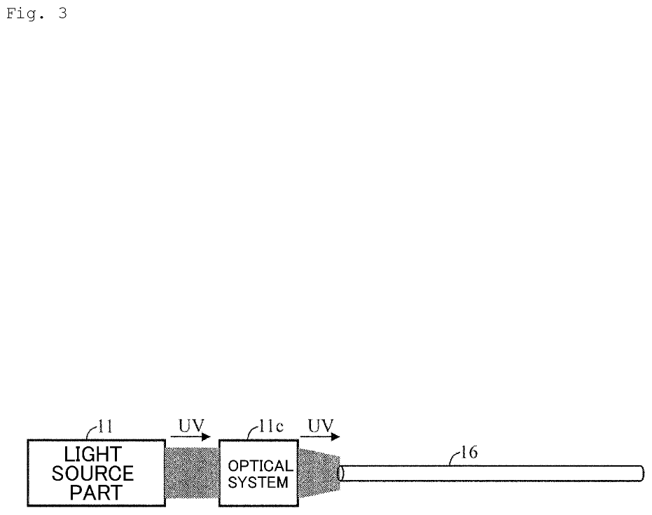

FIG. 7(A) illustrates the state of the optical transmission line 16 described with reference to FIG. 3. Since the optical transmission line 16 is a single-core optical fiber, only one core is used. Here, since the LED for generating ultraviolet light has a large light-emitting surface, the optical system 11c cannot collect light up to the diameter of the core 52, and reaches the diameter of the light collecting region Lc. Therefore, much ultraviolet light cannot enter the core 52, and much of the ultraviolet light power generated by the LED is wasted. In this way, it is difficult to cause ultraviolet light to efficiently enter the core 52 having a narrow area in the optical transmission line 16.

FIG. 7(B) illustrates the state of the optical transmission line 26 described with reference to FIG. 4. Since the optical transmission line 26 is a bundle optical fiber obtained by bundling a plurality of single-core optical fibers 51a, a plurality of cores 52 are also used. In FIG. 7(B), as an example, seven single-core optical fibers 51a are bundled in a hexagonal close-packed structure, and the number of cores is seven. Here, the optical system 11c is adjusted so that the plurality of cores 52 of the bundle optical fiber are included in the light collecting region Lc. In the case of a bundle optical fiber as illustrated in FIG. 7(B), the ultraviolet light which has been wasted in FIG. 7(A) can also enter the core 52, and the ultraviolet light can enter the core 52 more efficiently than in the optical transmission line 16 in FIG. 7 (A).

FIG. 7(C) illustrates the state of the optical transmission line 26 described with reference to FIG. 4. Since the optical transmission line 26 is the multi-core optical fiber 51b, a plurality of cores 52 are used. In FIG. 7(C), as an example, seven cores 52 are disposed in a hexagonal close-packed structure. Here, the optical system 11c is adjusted so that the plurality of cores 52 of the multi-core optical fiber 51b are included in the light collecting region Lc. In the case of a multi-core optical fiber as illustrated in FIG. 7(C), the ultraviolet light which has been wasted in FIG. 7(A) can also enter the core 52, and the ultraviolet light can enter the core 52 more efficiently than in the optical transmission line 16 in FIG. 7 (A).

In the optical transmission system 301 of the present embodiment, the system cost can be reduced by using an LED as the light source, and the waste of the output power of the light source can be reduced by receiving and transmitting the ultraviolet light of a beam diameter which cannot be narrowed down by the optical system with a plurality of cores.

### Embodiment 2

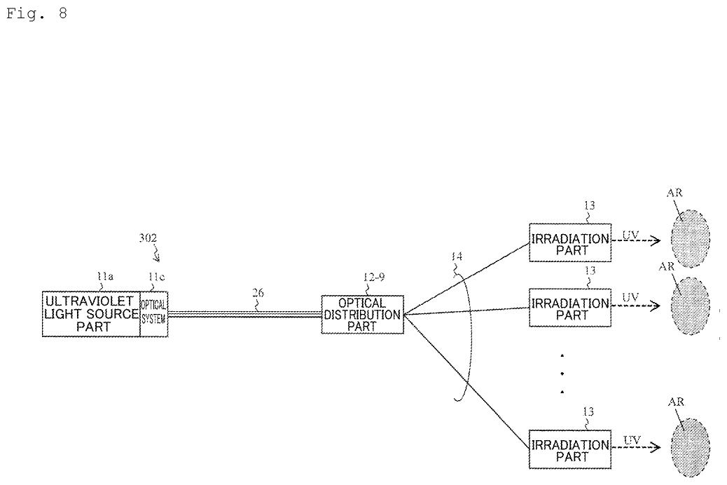

FIG. 8 is a diagram illustrating an optical transmission system 302 of the present embodiment. The optical transmission system 302 further includes irradiation parts 13 that irradiate a plurality of irradiation target regions AR with the ultraviolet light propagated through the plurality of cores, respectively, in comparison with the optical transmission system 301 illustrated in FIG. 4.

An optical distribution part 12-9 is disposed at the other end of the optical transmission line 26. The optical distribution part 12-9 outputs the ultraviolet light transmitted through each core of the optical transmission line 26 to a path 14 connected to each output port. Specifically, if the optical transmission line 26 is a bundle optical fiber, the optical distribution part 12-9 is a part where the bundled single-core optical fibers are disassembled, and the disassembled single-core optical fibers become the paths 14.

If the optical transmission line 26 is a multi-core optical fiber, the optical distribution part 12-9 is, for example, a fine-in/fan-out device disclosed in Reference 1. A multi-core optical fiber is connected to the fan-in side of the fine-in/fan-out device, and a path 14 is connected to the fan-out side.
(Reference 1) Fujikura Technical Journal No. 127 “Fan-in/fan-out device for multi-core optical fiber” (https://www.fujikura.co.jp/rd/gihou/backnumber/pages/_icsFil es/afieldfile/2015/02/24/127_R3.pdf), 2014, Vol. 2

The path 14 propagates the ultraviolet light distributed by the optical distribution part 12-9 to each irradiation part 13. The path 14 is a single-core optical fiber. Since the optical fiber is used, the optical fiber can be laid even in a small place where a robot and a device of the related art cannot enter. A single-core optical fiber having structures (1) to (5) in FIG. 6 can be used as the path 14.

The irradiation part 13 irradiates a predetermined target location (irradiation target region AR) to be sterilized or the like with the ultraviolet light transmitted through the path 14. The irradiation part 13 is constituted of an optical system such as a lens designed for the wavelength of ultraviolet light.

Since the optical transmission system 302 includes the optical distribution part 12-9 in comparison with the optical transmission system 301 of Embodiment 1, the system configuration of P-MP with a common light source can be achieved, and the system cost can be further reduced.

### Embodiment 3

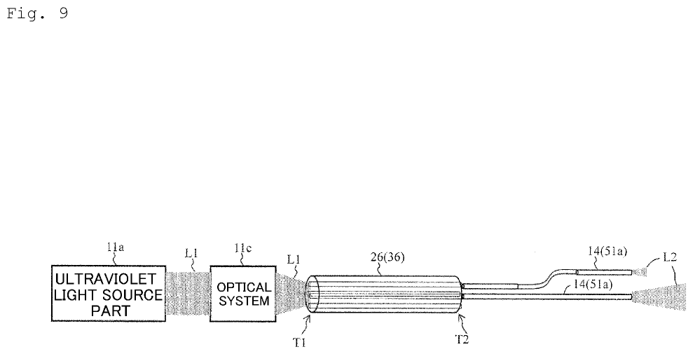

FIG. 9 is a diagram illustrating the problems when a bundle optical fiber 36 obtained by bundling a plurality of single-core optical fibers 51a is used as the optical transmission line 26 of the optical transmission system.

An optical transmission system that transmits ultraviolet light as light will be described.

(1) The optical transmission system has a point-to-multipoint configuration in which individual single-core optical fibers 51a bundled in the bundle optical fiber 36 are disassembled at the other end T2 (corresponding to the optical distribution part 12-9 in FIG. 8), and ultraviolet light is propagated to each irradiation target region. The ultraviolet light used for inactivating the irradiation target region must ensure safety because of a short wavelength that may cause damage to human skin and eyes. Human beings cannot visually check ultraviolet light, and there is a problem that it is difficult to check whether the ultraviolet light is being propagated through the disassembled single-core optical fiber 51a, that is, to ensure safety. This not only makes it impossible to ensure safety, but also has a problem that it is difficult to reduce costs because a separate device for ensuring safety must be prepared.

(2) Also, power deviation occurs at one end T1 of the bundle optical fiber 36 to which the ultraviolet light is coupled. Specifically, in the cross section of the bundle optical fiber 36, ultraviolet light having high power is propagated through the single-core optical fiber 51a near the center, and ultraviolet light having low power is propagated through the single-core optical fiber 51a at the outer peripheral portion. Therefore, the use of the single-core optical fiber 51a in the outer peripheral portion is limited to the use of the single-core optical fiber 51a near the center, and there is a problem that resources are wasted, such as not using propagated ultraviolet light depending on design. Moreover, even if there is ultraviolet light that is not used, it is difficult to reduce the power consumption of the ultraviolet light source part 11a, and as a result, there is also a problem that it is difficult to reduce the cost of the optical transmission system.

Therefore, the present embodiment has been made to solve the above problems, and an object thereof is to provide an optical transmission system capable of ensuring safety even when propagating ultraviolet light, and reducing costs by eliminating waste of resources.

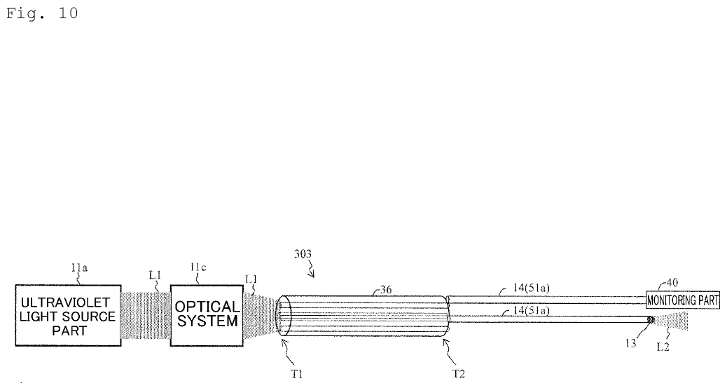

FIG. 10 is a diagram illustrating an optical transmission system 303 of the present embodiment that achieves the above object. The optical transmission system 303 further includes a monitoring part 40 in comparison with the optical transmission system 302 illustrated in FIG. 8. In the optical transmission system 302, the irradiation parts 13 are provided at the ends of all the paths 14, but in the optical transmission system 303, the end of at least one path 14 (the single-core optical fiber 51a disassembled at the other end T2) is not the irradiation part 13 but the monitoring part 40. The monitoring part 40 detects whether or not ultraviolet light is propagated through the single-core optical fiber 51a, or measures illuminance and power of the propagated ultraviolet light. That is, when the monitoring part 40 detects the ultraviolet light, it can be determined that the ultraviolet light propagates also to the other single-core optical fiber 51a and ultraviolet light L2 is output from the irradiation part 13.

The single-core optical fiber 51a monitored by the monitoring part 40 is preferably a single-core optical fiber 51a disposed on the outer periphery in the cross section of the bundle optical fiber 36. A power deviation when ultraviolet light from the light source part 11a as an LED is coupled at one end T1 of the bundle optical fiber 36 is used, the ultraviolet light having high power propagated through the single-core optical fiber 51a near the center part is used to inactivate an irradiation target region, and the ultraviolet light having low power propagated through the single-core optical fiber 51a near the outer peripheral portion is monitored by the monitoring part 40. With such a configuration, the ultraviolet light coupled to the single-core optical fiber 51a having low power is used for ascertaining the propagation state of the ultraviolet light without wasting the ultraviolet light, and safety can be ensured, and the system cost can be reduced without installing a new device for ensuring safety.

### Embodiment 4

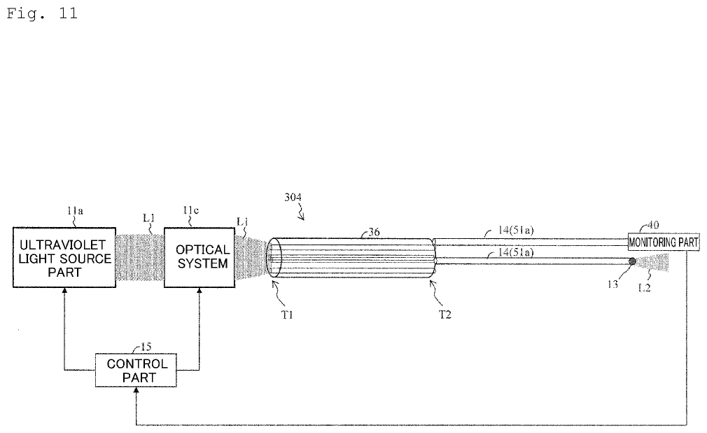

FIG. 11 is a diagram illustrating an optical transmission system 304 of the present embodiment. The optical transmission system 304 further includes a control part 15 that performs feedback control on at least one of power of the ultraviolet light L1 output from the light source part 11a and the coupling state adjusted by the optical system 11c so that power of the light monitored by the monitoring part 40 becomes a desired value in comparison with the optical transmission system 303 illustrated in FIG. 10.

The optical transmission system 304 also uses power deviation when ultraviolet light from the light source part 11a as an LED is coupled at one end T1 of the bundle optical fiber 36, uses the ultraviolet light having high power propagated through the single-core optical fiber 51a near the center part to inactivate an irradiation target region, and causes the monitoring part 40 to monitor the ultraviolet light having low power propagated through the single-core optical fiber 51a near the outer peripheral portion. With such a configuration, the ultraviolet light coupled to the single-core optical fiber 51a having low power is used for ascertaining the propagation state of the ultraviolet light without wasting the ultraviolet light, and safety can be ensured, and the system cost can be reduced without installing a new device for ensuring safety.

Further, the optical transmission system 304 feeds back monitoring information (whether or not ultraviolet light is propagating, or the power and illuminance of the ultraviolet light) in the monitoring part 40 to the control part 15. Communication from the monitoring part 40 to the control part 15 may be wired or wireless. The control part 15 can perform the following controls on the ultraviolet light source part 11a on the basis of the monitoring information.

(1) When the power of the ultraviolet light is greater than a desired value, an instruction is issued to suppress or stop the output of the ultraviolet light L1.

(2) When the power of the ultraviolet light is smaller than a desired value, an instruction is issued to increase the output of the ultraviolet light L1.

The control part 15 can also perform the following controls on the optical system 11c on the basis of the monitoring information.

(1) When the power of the ultraviolet light is greater than a desired value, an instruction is issued to increase the size of the spot shape of the ultraviolet light L1 (to decrease the illuminance at one end T1 and decrease the power of the ultraviolet light coupled to the single-core optical fiber 51a), to shift the optical axes of the ultraviolet light L1 and the bundle optical fiber 36, or to block it with a shutter, a filter, or the like.

(2) When the power of the ultraviolet light is smaller than a desired value, an instruction is issued to reduce the size of the spot shape of the ultraviolet light L1 (to increase the illuminance at one end T1 and increase the power of the ultraviolet light coupled to the single-core optical fiber 51a), to align the optical axes of the ultraviolet light L1 and the bundle optical fiber 36, or to open a shutter, a filter, or the like.

The optical transmission system 304 is provided with the control part 15, thereby ensuring the safety of the entire system. In addition, since the optical transmission system 304 can vary the power of the ultraviolet light to the irradiation target region, the irradiation time can be shortened according to the situation of the irradiation target region, and the waste of the ultraviolet light power can be reduced to save the power of the ultraviolet light source part 11a. In other words, the optical transmission system 304 enables cost reduction during system design and operation.

### Embodiment 5

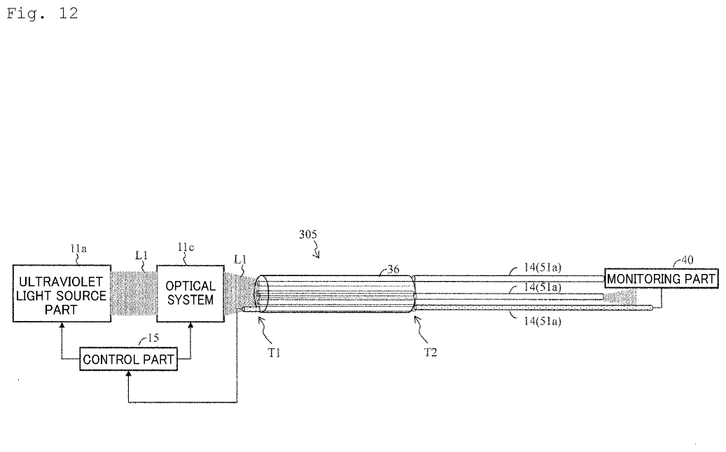

FIG. 12 is a diagram illustrating an optical transmission system 305 of the present embodiment. The optical transmission system 305 is different from the optical transmission system 304 illustrated in FIG. 11 in that the control part 15 performs the feedback control using at least one of the plurality of single-core optical fibers 51a of the bundle optical fiber 36. Here, the single-core optical fiber 51a used for the feedback control is preferably disposed on the outer periphery in the cross section of the bundle optical fiber 36.

The optical transmission system 305 connects the monitoring part 40 and the control part 15 by using those of the single-core optical fiber 51a included in the bundle optical fiber 36 that are not used for the propagation of the ultraviolet light and the monitoring of the monitoring part 40 for communication. Here, since one or a plurality of monitoring parts 40 may be provided, one or a plurality of single-core optical fibers 51a for communication may be provided.

Further, the single-core optical fiber 51a for monitoring ultraviolet light and the single-core optical fiber 51a for communication may be collectively installed and operated like a tape wire from the other end T2 to the monitoring part 40. Thus, since a communication line (an optical fiber cable or other communication means such as radio or the like) for feedback to the control part 15 is not separately prepared, the cost of the entire system can be reduced. In addition, when the irradiation target region and the monitoring part 40 are installed at the same location, by tape-forming the single-core optical fibers 51a for ultraviolet light propagation, ultraviolet light monitoring, and communication, the cost of system operation and management can be further reduced.

### Other Embodiments

In the above embodiments, the case where the ultraviolet light source part 11a is an LED has been described. However, in the present invention, the ultraviolet light source part 11a is not limited to an LED, but may be a light source (for example, an incandescent lamp or a discharge lamp) having the following optical characteristics.
There are variations in wavelength, amplitude, or phase.
Light scatters.
Spontaneous release.

Although the above-described embodiments have been described with reference to an example in which the optical transmission system includes an ultraviolet light source part, the present invention is not limited to ultraviolet light. The light L1 from the light source part may be infrared light or visible light.

Note that the “spot shape” described in this specification is a concept including the “beam diameter.”

### REFERENCE SIGNS LIST

11 Light source part
11a Ultraviolet light source part
11c Optical system
12, 12-9 Optical distribution part
13 Irradiation part
14 Path
15 Control part
16, 26 Optical transmission line
40 Monitoring part
51a Single-core optical fiber
51b Multi-core optical fiber
52 Core
52a Region
53 Hole
53a Hole group
53c Hole
60 Clad
300 to 305 Optical transmission system

1. An optical transmission system comprising:
a light source part configured to output light;
an optical transmission line configured to propagate the light through a plurality of cores of a multi-core optical fiber or a plurality of cores of a bundle optical fiber obtained by bundling a plurality of single-core optical fibers; and
an optical system configured to cause the light output from the light source part to enter the plurality of cores,
wherein the optical system is configured to arbitrarily adjust a coupling state in which the light enters the core.

2. The optical transmission system according to claim 1, wherein the optical system is configured to narrow down a spot shape of the light to a size including all of the plurality of cores in an optical axis direction as the adjustment of the coupling state.

3. The optical transmission system according to claim 1, further comprising irradiation parts configured to irradiate a plurality of irradiation target regions with the light propagated through the plurality of cores, respectively.

4. The optical transmission system according to claim 1, further comprising a monitoring part configured to monitor propagation of the light with respect to at least one of the cores.

5. The optical transmission system according to claim 4, wherein the core monitored by the monitoring part is the core disposed on an outer periphery in a cross section of the optical transmission line.

6. The optical transmission system according to claim 4, further comprising a control part configured to perform feedback control on at least one of power of the light output from the light source part and the coupling state adjusted by the optical system so that power of the light monitored by the monitoring part becomes a desired value.

7. The optical transmission system according to claim 6, wherein the control part is configured to perform the feedback control using at least one of the cores.

8. The optical transmission system according to claim 7, wherein the core used for the feedback control is the core disposed on an outer periphery in a cross section of the optical transmission line.

9. An ultraviolet light irradiation system comprising:
an ultraviolet light source part having a light emitting diode (LED) configured to output ultraviolet light;
an optical transmission line configured to propagate the ultraviolet light through a plurality of cores of a multi-core optical fiber or a plurality of cores of a bundle optical fiber obtained by bundling a plurality of single-core optical fibers; and
an optical system configured to narrow down a beam diameter of the ultraviolet light output from the LED to a diameter of a circle including all of the plurality of cores in an optical axis direction, and causes the ultraviolet light to enter the plurality of cores.

10. The ultraviolet light irradiation system according to claim 9, further comprising irradiation parts configured to irradiate a plurality of irradiation target regions with the ultraviolet light propagated through the plurality of cores, respectively.
